(19) United States
(12) Patent Application Publication
(10) Pub. No.: US 2007/0259330 A1
(43) Pub. Date: Nov. 8, 2007

(54) SEPARATION OF CELLS

(75) Inventors: Gregory Daryll Goddard, Ballston Spa, NY (US); Mohan Mark Amaratunga, Clifton Park, NY (US); Cyndy D. Sanberg, Spring Hill, FL (US); James van Alstine, Stockholm (SE); Oleg Kuzmenok, Tampa, FL (US)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, Uppsala (SE)

(21) Appl. No.: 11/739,890

(22) Filed: Apr. 25, 2007

Related U.S. Application Data
(60) Provisional application No. 60/797,645, filed on May 4, 2006.

(51) Int. Cl. A01N 1/02; C12N 5/08

(52) U.S. Cl. 435/2; 435/325; 435/372

(57) ABSTRACT

The present invention relates to the isolation of human stem cells for use e.g. in cell therapy. More specifically, the invention is a method of separating mononuclear cells from human blood, which method comprises providing a human blood sample together with density gradient media (DGM) in a container; spinning the container comprising blood and DGM; and collecting the DGM fraction that comprises mononuclear cells; wherein the DGM has a density which is >1.080 and <1.090 g/cm³ as measured at 25° C. The invention also relates to bags and tubes which contain density gradient media wherein the density and osmolality has been optimized for use in the present method, preferably as kits.

### CROSS-REFERENCE TO RELATED APPLICATIONS

This application claims priority to U.S. provisional patent application No. 60/797,645 filed May 4, 2006; the disclosure of which is incorporated herein by reference in its entirety.

### TECHNICAL FIELD

The present invention relates to the field of cell separation, and more specifically to a method of separating mononuclear cells from blood. The invention also encompasses a separation media which is used in the present method, a container filled with such media and a kit useful in cell separation.

### BACKGROUND OF THE INVENTION

Blood mononuclear cells, such as peripheral blood mononuclear cells (PBMC), are blood cells having a round nucleus, such as lymphocytes, monocytes and stem cells.

As is well known, lymphocytes and monocytes are critical components in the immune system to fight infection and adapt to intruders.

Stem cells on the other hand are immature subpopulations of cells that have the potential to differentiate into a wide variety of specialized cell types such as bone, muscle, pancreas, liver, or blood cells. These undifferentiated cells have the ability of self-renewal which preserves their continuous supply. Embryonic stem cells (ESCs) are commonly derived from 4- to 5-day-old embryos. At this stage, the embryos are spherical and are known as blastocysts. Each blastocyst consists of 50 to 150 cells and includes three structures: an outer layer of cells, a fluid-filled cavity, and a group of about 30 pluripotent cells at one end of the cavity. This latter group of cells called the inner cell mass, form all the cells of the body. Adult stem cells on the other hand are undifferentiated cells that are found in small numbers in most adult tissues. However, they are also found in children and can be extracted from umbilical cord blood. The primary roles of adult stem cells in the body are to maintain and repair the tissues in which they are found. They are usually thought of as multipotent cells, giving rise to a closely related family of cells within the tissue. An example is haematopoietic stem cells, which form all the various cells in the blood. Haematopoietic stem cells are currently of interest in research, as they can differentiate into neurons, glia, skeletal muscle cells, heart muscle cells, and liver cells.

Blood from the placenta and umbilical cord that are left over after birth is a rich source of haematopoietic stem cells. These so-called umbilical cord stem cells have been shown to be able to differentiate into bone cells and neurons, as well as the cells lining the inside of blood vessels.

In cell therapy, the idea is to use adult stem cells from the patient to be treated, and to expand said cells in culture, treat them to differentiate into the desired cells, and then to reintroduce them into the patient. The use of the patient's own cells would eliminate any possibility that they might be rejected by the immune system.

The most commonly used technique to isolate peripheral blood mononuclear cells (PBMNC) is to centrifuge whole blood over an isoosmotic barrier often denoted density gradient media (DGM). Density gradient media are commercially available products, such as Ficoll-Paque™ (GE Healthcare), which is an established reagent for cell separation in peripheral blood and bone marrow. Ficoll-Paque™ (GE Healthcare), which is obtainable in a density of 1.077 g/cm³. However, the cell composition in cord blood is significantly different from that of peripheral blood and marrow. Thus, there is still a need of a more specialized DGM, which has been optimised for the separation of certain cell types present in cord blood.

Histopaque™-1077 (Sigma-Aldrich) is another commercially available DGM product. More specifically, Histopaque™-1077 is promoted for the isolation of mononuclear cells and in vitro diagnostics. The density is 1.077±0.001 g/ml. In the application note to this product, it is stated to be capable of providing viable, mononuclear cells from small blood volumes. The procedure described for this product is according to the note suitable for the study of cell mediated lymfolysis and HLA typing. However, there is still a need in this field of a more optimised DGM, which provides mononuclear cells from blood in yields useful for clinical applications.

A similar solution is HISTOPAQUE®-1077, adjusted to a density of 1.083 g/mL, which is also available from Sigma-Aldrich which is promoted as aseptically filled. According to the specification sheet, its osmolality is in the range 297-325 Osm/kg H₂O. It is stated to facilitate recovery of viable mononuclear cells from rats, mice, and other mammals. Similar to the other Histopaque™product, it appears to be suitable for small volumes of blood. However, it is well known that human stem cells differ substantially in terms of many properties from those of other mammals. Thus, for human clinical applications, there is still a need in this field of a DGM, which is capable of providing sufficiently high yields of mononuclear cells from human blood, such as human cord blood.

U.S. Pat. No. 5,474,687 (Activated Cell Therapy) relates to the enrichment of CD34⁺ cells. More specifically, a method is disclosed, which comprises

layering a cell mixture containing CD34⁺ cells into a centrifuge tube, said density gradient solution having an osmolality of 280±10 mOsm/kg H₂O and a specific density within 0.0005 g/ml of the specific density of said CD34⁺ cells;

centrifuging said tube at a gravitational force sufficient to pellet cells having specific densities greater than the specific density of the density gradient material in said tube; and

collecting from the upper portion of said tube an enriched population of CD34⁺ cells.

The tube used in the method comprises an annular member disposed in said tube and defining an opening there through, which opening has an area less than the area of a cross section of the tube. In one embodiment, the method further comprises incubating said cell mixture with a cell type-specific binding agent linked to carrier particles prior to centrifugation, said particles having a specific density that is at least 0.001 g/ml greater than the specific density of said density gradient solution. This binding agent may bind to non-CD34⁺ cells, and may e.g. be an antibody directed to the CD45 antigen. The density gradient solution may e.g. be selected from the group consisting of Percoll™, Ficoll™, Ficoll-Hypaque™, albumin, sucrose and dextran.

As appears from the above, there is still a need in this field of novel purification protocols which allow efficient purification of viable mononuclear cells from blood in yields useful for clinical applications.

### BRIEF DESCRIPTION OF THE INVENTION

One aspect of the present invention is to provide a method of separating viable mononuclear cells (MNC) from human blood in yields useful for in vivo applications. This can be achieved by a method as defined in the appended claims.

A specific aspect is a method which provides mononuclear cells from human cord blood, which has been thawed following cryopreservation, using the method according to the invention.

Another aspect is a method as described above, which results in a fraction of viable mononuclear cells wherein the red blood cell (RBC) contamination is reduced substantially or even eliminated.

A further aspect of the present invention to provide a density gradient media which is optimized for the separation of mononuclear cells from cord blood, such as cryopreserved cord blood. This aspect may be a sucrose-based optimised media as defined in the appended claims.

A specific aspect is such a density gradient media which is provided in a suitable container, such as a tube or a plastic bag. A tube comprising optimised density gradient media according to the invention may contain a dividing part to separate the blood from the sample. A plastic bag comprising optimised density gradient media according to the invention may be provided as a kit suitable for use with a centrifuge or automated instrument for cell separation and/or processing.

In an advantageous aspect, the density gradient media, and the tubes, bags or kits according to the invention have been sterilized.

Further aspects and advantages of the present invention will appear from the detailed description that follows below.

### BRIEF DESCRIPTION OF THE DRAWINGS

FIG. 1 shows a comparison of the MNC cell absolute number (yield from the one hUCB unit) after using different Ficoll-Paque gradient densities. Values are presented as the mean±SD. (ANOVA test two population). *p<0.05 compared to viability obtained by Ficoll 1.083 density.

FIG. 2 shows the viability of cells measured by using Vi-cell counter. *p<0.05 compared to viability obtained by Ficoll 1.083 density.

FIG. 3 shows the cell diameter measured by using Vi-cell counter. **p<0.01 compared to cell diameter obtained by Ficoll 1.077 g/cm³ density.

FIG. 4 shows a comparison of the volumes of whole blood (millilitres). Values are presented as the mean±SD. n.s. no statistical significance vs. 1.083 g/cm³ (ANOVA test two population).

FIG. 5A and FIG. 5B show a comparison of the viable cell number between pre-freeze samples (FIG. 5A) and post-thaw samples (FIG. 5B). As appears from the figure, the density gradient media of 1.083 g/ml is superior to the other tested densities.

FIG. 6A, FIG. 6B and FIG. 6C show the results of characterization of CD45+, CD34+ and CD133+ cells using flow cytometry, results presented as cell number.

### DETAILED DESCRIPTION OF THE INVENTION

In a first aspect, the present invention relates to a method of separating mononuclear cells from blood, which method comprises providing a human blood sample together with density gradient media (DGM) in a container; spinning the container comprising blood and DGM; and collecting the DGM fraction that comprises mononuclear cells; wherein the DGM has a density which is >1.080 and <1.090 g/cm³ as measured at 25° C.

In an advantageous embodiment, the density of the DGM is about 1.083 g/cm³, such as 1.083±0.003 g/cm³ as measured at 25° C. As known in this area, the density of a density gradient media will vary with temperature, and consequently needs to be specified by the temperature at which it was measured. For example, the above-mentioned advantageously used DGM presents a density of 1.0845 at 20° C.

In the general aspect, the blood from which mononuclear cells are separated may originate from any human source such as peripheral blood, embryonic blood, placental blood or umbilical cord blood. In a specific embodiment, the blood sample originates from cord or placenta. In one embodiment, the blood originates from human bone marrow, which has been processed according to well known methods into a form suitable for density gradient separation. In one embodiment, the mononuclear cells separated are characterized as CD34+ cells. In an advantageous embodiment, the present method is used to prepare a purified fraction of mononuclear cells for in vivo use, such as in cell transplantation or cell therapy.

As is well known, cord blood is frequently collected after birth, cryopreserved and stored for a period of time and then thawed to be used in the clinic or research lab. Thus, in one embodiment, the blood has been cryopreserved and thawed before the separation. In an advantageous embodiment, irrespective of whether it has been thawed or not, the blood is heated to a temperature close to room temperature, which is a suitable temperature to carry out the method of the invention.

In an advantageous embodiment of the present method, the DGM is comprised of neutral, highly branched, hydrophilic polymers of sucrose. In a more advantageous embodiment, the osmolality of the DGM is >300 Osm/kg H₂O, such as >325 Osm/kg H₂O. In an advantageous embodiment, the osmolality is in the range 325-350, such as 330-350 or 330-340 Osm/kg H₂O. The DGM will be discussed in more detail below. In an alternative embodiment, the DGM used in the present method is comprised of iodixanol in water presenting the herein defined density and preferably the osmolality above.

In one embodiment of the present method, the blood sample has been diluted before the separation. Such dilution is advantageously carried out with a suitable buffer, such as a salt e.g. a phosphate buffer; a salt solution such a Hank's balanced; or cell culture medium. As the skilled person will appreciate, if and how the dilution is carried out will depend on the contents of mononuclear cells in the blood and the volume used. Further, if required, anti-coagulant may be added. Again, the skilled person in this field will be able to decide in which cases and which amount anti-coagulant can be added. In an advantageous embodiment, the total volume of the blood and DGM is 10-200 ml, such 20, 50 or 100 ml. In a specific embodiment, the total volume of the blood and DGM is above 200 ml.

In an advantageous embodiment, the blood sample is layered on top of the DGM in the container. In an advantageous embodiment, the container is a tube or a bag, as will be discussed in more detail below.

In the most advantageous embodiment, the spinning of blood sample with DGM is achieved by centrifugation. In a specific embodiment, the centrifugation is carried out at a speed of 400×G, and may last for about half an hour. Any commonly used centrifuge may be used, such as a temperature-controlled centrifuge.

As the skilled person will understand, the centrifugation of blood sample and DGM will result in an upper plasma fraction, and a DGM layer containing the mononuclear cells underneath. Red blood cells, which are regarded contaminants in the present method, will gather at the bottom of the container. Thus, in one embodiment, the desired monocyte-containing DGM fraction is recovered by aspiration after removal of the plasma fraction. The aspiration may be carried out with a commonly used syringe, or with an automated instrument. Preferably, the aspiration is carried out under sterile or aseptic conditions.

A specific aspect of the present method is a method of purifying lymphocytes from erythrocytes, thrombocytes and granulocytes in a blood sample, which method comprises a method according to the invention, as discussed above, and an additional step of isolating said lymphocytes from the DGM fraction. Lymphocytes have a number of roles in the immune system, including the production of antibodies and other substances that fight infection and diseases. Thus, lymphocytes isolated according to the present invention are useful e.g. for clinical and diagnostic use.

Another embodiment of this aspect is a method of purifying monocytes from erythrocytes, thrombocytes and granulocytes in a blood sample, which method comprises a method according to the invention and an additional step of isolating said monocytes from the DGM fraction. As is well known, a monocyte is a specific type of white blood cell, and like the isolated lymphocytes, monocytes isolated according to the invention are useful e.g. for clinical and diagnostic use.

A further embodiment of this aspect is a method of purifying stem cells from erythrocytes, thrombocytes and granulocytes in a blood sample, which method comprises a method according to the invention and an additional step of isolating said stem cells from the DGM fraction. As is well known, stem cells are the cells from which other types of cells develop, and may be embryonic or human. Stem cells isolated according to the invention are useful for in vitro and/or in vivo purposes, such as for research, clinical and diagnostic use. In an advantageous embodiment, the present stem cells are used in vivo, and more specifically for transplantation purposes into patients. Such transplantation may e.g. be a method of replacing immature blood-forming cells that were destroyed, such as by cancer treatment. In this case, the stem cells are given to the person after treatment to help the bone marrow recover and continue producing healthy blood cells.

This embodiment is equally useful for the isolation of progenitor cells for in vitro and/or in vivo purposes. In this context, the term progenitor cell is used for immature or undifferentiated cells, typically found in post-natal animals. While progenitor cells share many common features with stem cells, the term is less restrictive.

The cells purified according to the present invention are useful in the context of cell therapy, such as in research related to cell therapy and/or for clinical or pre-clinical applications.

In a second aspect, the present invention relates to a density gradient media (DGM) as such. More specifically, the DGM according to the invention is comprised of neutral, highly branched, hydrophilic sucrose polymers, which DGM present a density as discussed above, such as in the range of 1.080-1.090 g/cm³, and an osmolality as discussed above, such as >325 Osm/kg H₂O, preferably in the range of 325-350, such as 330-350 or 330-350 Osm/kg H₂O. The DGM according to the invention may be prepared starting from a commercially available sucrose-based DGM, such as Ficoll-Hypaque™ (GE Healthcare, Uppsala, Sweden) or HistoPaque™ (Sigma-Aldrich), by careful modification of density and optionally also osmolality. In an advantageous embodiment, the DGM according to the invention is of GMP quality.

In a third aspect, the present invention relates to a container containing DGM and useful in the method according to the invention. Thus, in one embodiment, the container is a bag comprising the DGM according to the invention, which bag is made from synthetic polymers, preferably a plastic, e.g. a plastic laminate. In an advantageous embodiment, the bag is sterilizable. Thus, the bag may be sterilized separately and filled under aseptic conditions with sterile DGM, or more conveniently, the bag is filled with and sterilized with its DGM contents. In another embodiment, the container is a tube comprising the DGM according to the invention. The tube may be sterile and/or sterilizable, as the bag above. Further, the tube may contain some kind of physical partition means such as a horizontal wall to prevent mixing of the blood with the DGM. Such partition means have been described see e.g. U.S. Pat. No. 4,917,801.

In a fourth aspect, the invention relates to a kit for the purification of mononuclear cells from cord blood, which kit comprises a bag or a tube, which contains density gradient media according to the invention having a density as discussed above, such as in the range of 1.080-1.090 g/cm³, and an osmolality as discussed above, such as >325 Osm/kg H₂O, preferably in the range of 325-350, such as 330-350 or 330-350 Osm/kg H₂O. In one embodiment, the present kit is comprised of a container containing DGM comprised of neutral, highly branched, hydrophilic sucrose polymers, which DGM present a density in the range of 1.083±0.003 g/cm³ and an osmolality >325 Osm/kg H₂O. In one embodiment, the kit is sterile and optionally adapted for use in an automated cell processing instrument. In one embodiment, the kit comprises instructions for use, preferably for use in a method of separating human mononuclear cells from blood such as cord blood, placenta blood or blood marrow.

### EXAMPLES

The present examples are provided for illustrative purposes only, and should not be interpreted in any way as limiting the scope of the invention as defined by the appended claims. All references provided below and elsewhere in the present specification are hereby included herein via reference.

### Materials and Methods

Cord blood (CB) Collection. Umbilical cord blood, which is a rich source of stem and progenitor cells, was obtained by direct drainage from the cord and/or by needle aspiration from the delivered placenta at the root and distended veins. Umbilical cord blood was collected from delivered placentas with syringes containing an anticoagulant, citrate phosphate dextrose (CPD) (CPD:blood 1:12).

### Processing CB or Standard Operating Procedure HUCB Processing.

Isolation of Mononuclear Cells (MNC). The blood bag contents were mixed by gentle rotation for approximately 30 seconds to ensure that the contents were mixed well. The tubing was clamped approximately 2 inches from the opening of the bag with a sterile hemostat. The tubing was sterilized above the hemostat with alcohol wipes. The sterilized tubing was held with a hemostat, and the tubing was cut using sterile scissors approximately 2 inches above the hemostat. The blood was aliquoted into sterile, labelled, 50 mL conical tubes (25 mL of blood/tube) and the volume of the cord blood without anticoagulant was calculated by subtracting the amount of anticoagulant. DPBS (Dulbecco's Phosphate-Buffered Saline, pH 7.2-7.4, Cellgro) was added to each labelled tube of cord blood, to a volume of 35 mL and the tubes were inverted carefully 2 or 3 times. Each tube was underlaid, using a 10 mL sterile pipette, with diluted cord blood and 12.8 mL of Histopaque-1077 (Sigma-Aldrich, #10771., St. Louis, Mo.) and centrifuged at 400×g for 30 minutes at 22° C. The plasma was carefully removed approximately 1.5 cm above the MNC layer and stored in clean 50 mL tubes. The MNC layer was removed using a 10 mL sterile pipette. The cells were transferred into 50 mL tubes and RPMI was added to a volume of 45 mL. The tubes were centrifuged at 400×g for 15 minutes at 22° C. The supernatant was decanted and the cell pellets were resuspended by the addition of 5 mL of RPMI to each tube, and adjusting the total volume to 45 ml and centrifugation at 400×g for 10 minutes at 22° C. The supernatant was decanted and the cell pellet carefully resuspended in RPMI and then transferred to a sterile 15 mL conical tube. The tube was mixed gently by inversion. The tubes were centrifuged at 400×g for 10 minutes at 22° C. The remaining supernatant was discarded being careful not to disturb the cell pellet. 750 μL of plasma was added to resuspend the cell pellet. Once the cells were completely in suspension, the volume was aliquoted to 1 ml using a serological pipette and stored on ice. 20 μl of cell suspension was transferred to the Vi-Cell Viability Analyzer (Beckman Coulter) for cell counting and viability. Once the viable cell count had been determined by the Vi-Cell, the viable cell count number was typed into a cell solution program (MS Xcel), press enter, and this program calculated all volumes of reagents (90% autologous plasma, 10% DMSO) to be added to the final volume of the cell suspension prior to aliquoting into cyrovials. This program also determined the number of Research vials and 1 Quality Assurance (QA) vial based on a storage volume of 20 million cells per vial. In addition, 1 Archive (AR) vial was stored for each sample. The Archive (AR) vial contained the remainder of the cells after the Research and QA vials have been aliquoted. The program also determined the concentrations of reagents for the AR vial which contained a separate cell suspension since the total amount of cells present in this AR was much lower than the Research and QA vials. All contents within each vial were aliquoted properly and completely before the samples were frozen. The cryovials were placed in a rack, in a controlled rate freezer, and frozen using the assigned pre-set profile #1.

Human umbilical cord blood (HUCB) thawing. The cryovials were removed (Corning #430488) from the liquid nitrogen container and placed in a 37° C. water bath for 5 min. The thawed cell suspension was rapidly transferred from the cryovial to a 15-mL conical centrifuge tube (Falcon, #352057) containing 10 mL of DPBS and centrifuged at 400×g for 10 minutes at +21° C. The supernatant was removed without disturbing the cell pellet and 10 mL of DPBS was added to resuspend the cells again. After centrifugation, the pellet was resuspended in 1 ml of the DPBS, and 10 μl of HUCB suspension was removed for counting the cells using a hemacytometer or Vi-Cell Analyzer.

Blood smears for Giemsa. The blood samples were obtained from the fresh HUCB and were taken for morphological analysis. The smears were dried for 30 min, fixed in methanol for 7 min, then stained by Giemsa (Sigma-Aldrich, GS80, St. Louis, Mo.) as previously described (Brown and Febiger, 1993). After staining, blood smears were rinsed several times in distilled water and cover-slipped with Permount (Fisher Scientific, Fair Lawn, N.J.). The morphology of the peripheral blood cells was examined under an Olympus BX-60 microscope. The images were analyzed by Image-Pro Plus version 4.1 for Windows software (Silver Spring, Md.). Analyses for CBC (complete blood count) and white blood cell differential (WBC) were performed by Antech Diagnostics (NY, USA). The blood smears were treated as disclosed in Brown A, and L. Febiger, Hematology: Principles and Procedures (6th ed.), Lea and Febiger, Philadelphia (1993), p. 101.

Flow Cytometry. Surface antigens were detected by double-color immunofluorescence assay combining fluorescein isothyocyanate (FITC) or phycoerythrin (PE) conjugated monoclonal antibodies (Mab). These included: CD45-FITC/CD34-PE (#341070), Isotype Control (Mouse IgG1-FITC, #349041) (Becton Dickinson, BD), and CD133 (#130-090-422, MACS). All the Mab were used at the concentrations titrated for optimal staining. Treatment of leukocyte with Mab was done according to the manufacturer's recommendations. Irrelevant fluorochrome-conjugated murine isotype-matched Mab were always included in the staining protocols as a negative control. Detection of fluorescence of stained cells was performed with a FACScan flow cytometer (BD) equipped with Argon laser tuned to 488 nm. Calibration beads were used for monitoring and optimizing the instrument settings. Data were acquired with LYSIS II software (BD). Forward light scattering (FCS), orthogonal light scattering (SSC), and fluorescence signals (FL-1-FITC, FL-2-PE) were sorted in listmode data files. For data standardization gated acquisition of living lymphocyte population was performed routinely. A minimum of 50,000 cells were analyzed and at least 5,000 gated events were measured for each sample. All data were analyzed using PAINT-A-GATE software (Becton Dickinson).

### DGM Conditions

Density gradient media (DGM) according to the invention were prepared in different densities and osmolalities by modification of Ficoll-Paque™ (GE Healthcare Bio-Sciences, Uppsala, Sweden), which is comprised of neutral, highly branched, hydrophilic polymers of sucrose. In the present experimental part and drawings, “Ficoll” refers to “Ficoll-Paque”™. In the present application, commercial products used for comparative purposes have been presented with the density provided by the supplier, while the density gradient media adapted by the present inventors presents a density of 1.083 as measured at 25° C.

Standard industrial protocol was used in all the experiments below, unless otherwise stated, to separate MNC fraction from human umbilical cord blood.

| Sample | | Volume | | Density | Osmolality |
|---|---|---|---|---|---|
| Name | Batch ID | (L) | pH | (g/cc) | (mmol/kg) |
| FPP 1076 | 110905A | 1 | 7.12 | 1.0757 | 274 |
| FPP 1080 | 111605A | 1 | 7.12 | 1.0804 | 307 |
| FPP 1083 | 111605A | 2 | 7.12 | 1.0832 | 317 |
| FPP 1090 | 111605A | 2 | 7.12 | 1.0903 | 343 |

### Example 1

### Pre-Freeze Samples

### Density of Samples

Density of FPP and number of CB samples used with each density:
i. 1.077, N=12
ii. 1.080, N=10
iii. 1.083, N=8
iv. 1.090, N=5

### Analysis

All assessments were done prior to freezing.

### Results

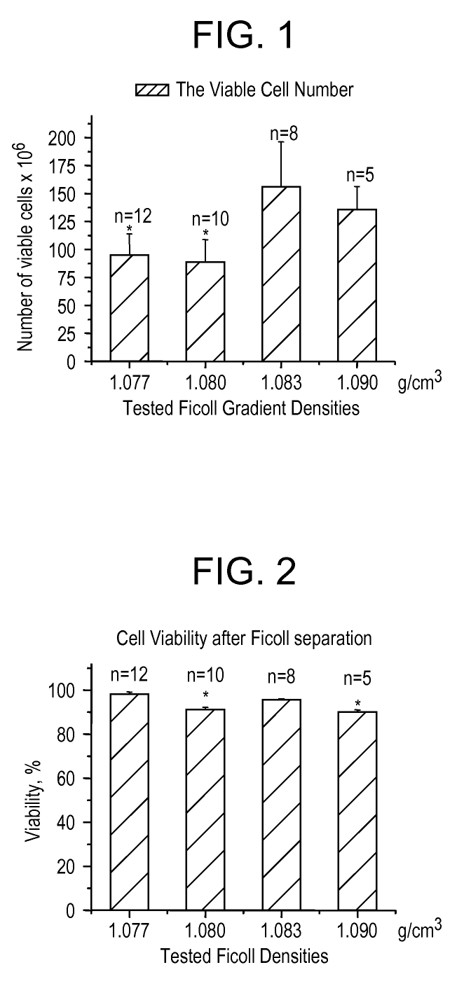

MNC Yield—no significant differences were observed between densities 1.083 and 1.090 (FIG. 1).

MNC Yield—densities 1.077 and 1.080 had a significantly lower number of cells as compared to 1.083 (FIG. 1).

Cell Viability—no significant differences were observed between densities 1.083 and 1.077 (FIG. 2).

Cell Viability—densities 1.080 and 1.090 exhibited significantly lower cell viability as compared to 1.083 (FIG. 2).

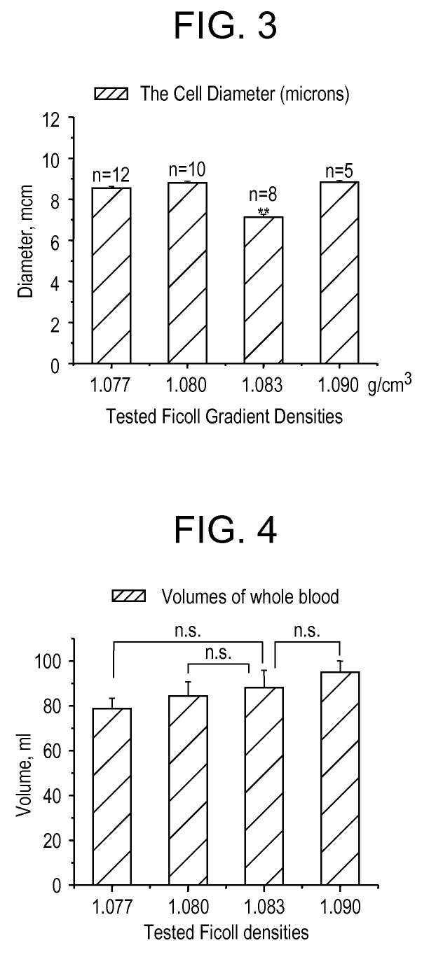

Cell Diameter—all cell diameters were compared to the standard 1.077 density (FIG. 3).

Density 1.083 had a significantly lower cell diameter as compared to 1.077 Note: this could be due to an increase of platelets or higher % of progenitor cells

Comparison of Unit Volumes—all unit volumes were averaged and compared for each density tested (FIG. 4).

No significant differences were observed between any of the densities. This should rule out any chance of volume variability affecting the MNC outcome.

### Example 2

### Post-Thaw Samples

### Density of Samples

Density of FPP and number of CB samples used with each density:
1.077, N=7
1.080, N=6
1.083, N=7
1.090, N=3

### Analysis

All assessments in this example were done post-thaw.

### Viability

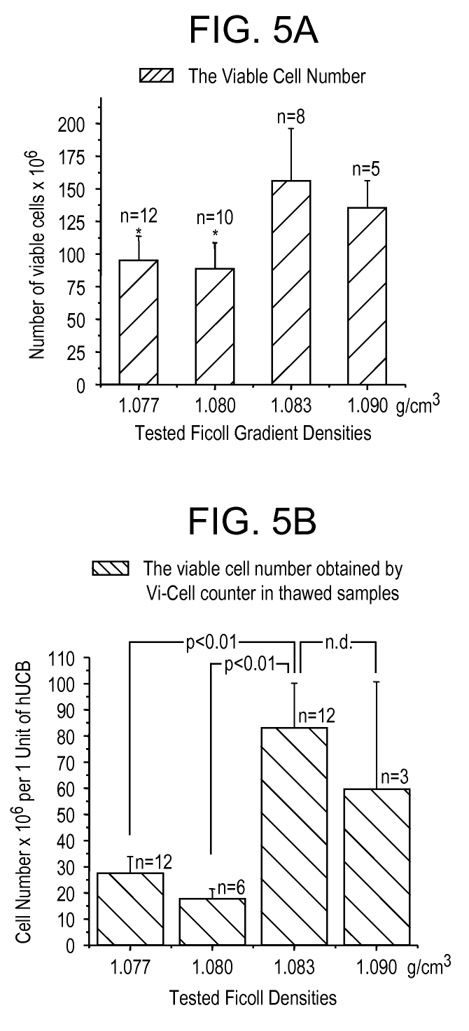

In this section, the MNC yield was compared between Pre-Freeze and Post-thaw samples (see FIG. 5A and B).

### Results

(A) Pre-freeze MNC Yield—densities 1.077 and 1.080 had a significantly lower number of cells as compared to 1.083.

(B) Post-thaw MNC Yield—1.083 density produced a significantly higher cell number than densities 1.077 and 1.080.

### Characterization

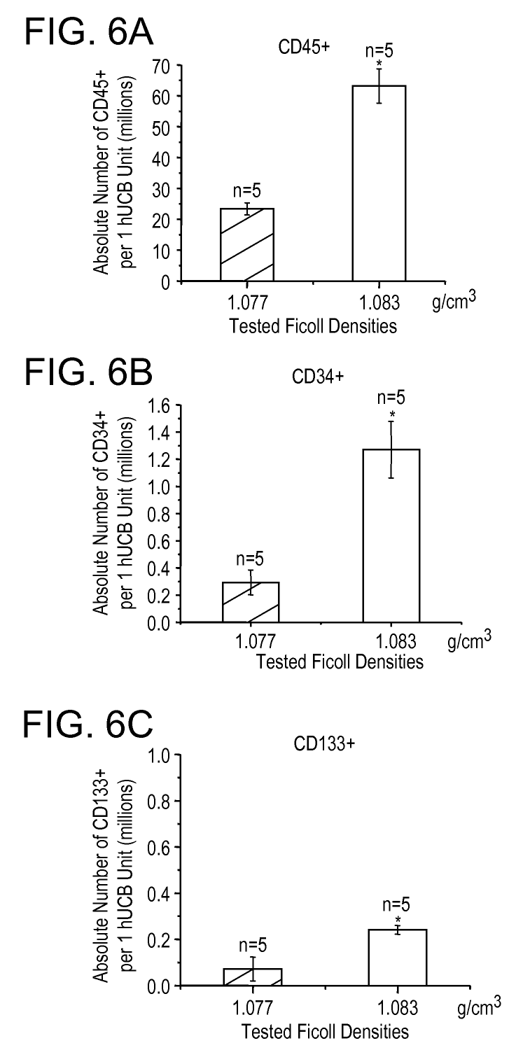

In this section, cells were characterized by Flow Cytometry—cell number (see FIG. 6A, B. and C). The cell number was calculated by measuring the number of viable cells post-thaw per unit, and then multiplying those cell numbers by the percentage for each density (data not shown).

### Results

(A) the number of CD45+ cells was significantly higher with 1.083 than 1.077, 63.17×10⁶ and 23×10⁶ cells respectively.

(B) the number of CD34+ cells was significantly higher with 1.083 than 1.077, 1.27×10⁶ and 0.294×10⁶ cells respectively.

(C) the number of CD133+ cells was significantly higher with 1.083 than 1.077, 0.2407×10⁶ and 0.072×10⁶ cells respectively.

The red blood cell (RBC) contamination was determined using Giemsa pre-Freeze and post-Thaw, N=5 for each density.

### Results

No significant differences were observed pre-freeze between densities 1.077 and 1.083, and no significant differences were observed post-thaw between densities 1.077 and 1.083.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

What is claimed is:

1. A method of separating mononuclear cells from blood, which method comprises providing a human blood sample together with density gradient media (DGM) in a container; spinning the container comprising blood and DGM; and collecting the DGM fraction that comprises mononuclear cells; wherein the DGM has a density which is >1.080 and <1.090 g/cm³ as measured at 25° C.

2. The method of claim 1, wherein the DGM has a density of 1.083 g/cm³.

3. The method of claim 1, wherein the blood sample is umbilical cord blood or placenta blood.

4. The method of claim 1, wherein the blood sample originates from bone marrow.

5. The method of claim 1, wherein the blood sample has been cryopreserved and thawed before the separation.

6. The method of claim 1, wherein the DGM is comprised of neutral, highly branched, hydrophilic polymers of sucrose.

7. The method of claim 1, wherein the blood has been diluted before the separation.

8. The method of claim 1, wherein the total volume of the blood and DGM is 10-200 ml.

9. The method of claim 1, wherein the blood sample is layered on top of the DGM in the container in said providing step.

10. The method of claim 1, wherein the container is a tube or a bag.

11. The method of claim 1, wherein the spinning is centrifugation.

12. The method of claim 1, wherein the mononuclear cell-containing DGM fraction is recovered by aspiration after removal of the plasma fraction.

13. The method of claim 1, wherein the DGM fraction comprises stem cells;
lymphocytes; and monocytes.

14. A method of purifying lymphocytes from erythrocytes, thrombocytes and granulocytes in a blood sample, which method comprises the method of claim 1 and an additional step of isolating said lymphocytes from the DGM fraction.

15. A method of purifying monocytes from erythrocytes, thrombocytes and granulocytes in a blood sample, which method comprises the method of claim 1 and an additional step of isolating said monocytes from the DGM fraction.

16. A method of purifying stem cells from erythrocytes, thrombocytes and granulocytes in a blood sample, which method comprises the method of claim 1 and an additional step of isolating said stem cells from the DGM fraction.

17. A density gradient media (DGM) comprised of neutral, highly branched, hydrophilic sucrose polymers, which DGM present a density in the range of 1.080-1.090 g/cm³ and an osmolality >325 Osm/kg H₂O.

18. A bag comprising the DGM of claim 17, which bag is made from synthetic polymers.

19. The bag of claim 18, which is sterilizable.

20. A tube comprising the DGM of claim 17.

21. A kit for the purification of mononuclear cells from human cord blood, which kit comprises the bag of claim 18 and instructions for its use.

22. The kit of claim 21, which is sterile.

23. The kit of claim 21, which is for use in vivo.

24. The DGM of claim 17, which present a density of about 1.083 g/cm³ and an osmolality of 325-350 Osm/kg H₂O.

25. The bag of claim 18, which is made of a plastic.

26. A kit for the purification of mononuclear cells from human cord blood, which kit comprises the tube of claim 20 and instructions for its use.

27. The kit of claim 26, which is sterile.

28. The kit of claim 26, which is for use in vivo.
